(19) United States
(12) Patent Application Publication
(10) Pub. No.: US 2008/0032937 A1
(43) Pub. Date: Feb. 7, 2008

(54) Bioavailability and Improved Delivery of Alkaline Pharmaceutical Drugs

(76) Inventors: Ruey J. Yu, Chalfont, PA (US); Eugene J. Van Scott, Abington, PA (US)

Correspondence Address: GOODWIN PROCTER LLP, 901 NEW YORK AVENUE, N.W., WASHINGTON, DC, 20001, US

(21) Appl. No.: 11/844,865

(22) Filed: Aug. 24, 2007

Related U.S. Application Data
(62) Division of application No. 10/792,273, filed on Mar. 4, 2004.
(62) Division of application No. 11/228,230, filed on Sep. 19, 2005.
(60) Provisional application No. 60/452,557, filed on Mar. 7, 2003.

(51) Int. Cl. A61K 31/7004; A61K 31/135; A61K 31/19; A61K 31/401; A61P 17/00; A61K 31/4164; A61K 31/56

(52) U.S. Cl. 514/23; 514/177; 514/396; 514/398; 514/399; 514/423; 514/557; 514/648

(57) ABSTRACT

Embodiments of the invention relate to a composition, a process of making the composition, and to the use of the composition. The compositions include a molecular complex formed between an alkaline pharmaceutical drug and at least one selected from a hydroxyacid, a polyhydroxy acid, a related acid, a lactone, or combinations thereof. The compositions provide improved bioavailability and improved delivery of the drug into the cutaneous tissues.

### RELATED APPLICATIONS

This application is a divisional of U.S. patent application Ser. No. 10/792,273 entitled: “Bioavailability and Improved Delivery of Alkaline Pharmaceutical Drugs,” filed on Mar. 4, 2004 which claims priority under 35 U.S.C. §119 to Provisional Patent Application No. 60/452,557, filed on Mar. 7, 2003, the disclosures of each of which are incorporated by reference herein in their entireties.

### BACKGROUND OF THE INVENTION

1. Field of the Invention

Embodiments of the invention relate to a process of making and the use of topical compositions including a molecular complex formed between an alkaline pharmaceutical drug and at least one selected from a hydroxyacid, a polyhydroxy acid, related acid, a lactone, or combinations thereof. The compositions provide improved bioavailability and improved delivery of the drug into the cutaneous tissues. The alkaline pharmaceutical drugs preferably are organic compounds that contain at least one amino, imino and/or guanido group in the molecules. The hydroxyacids, polyhydroxy acids, related acids, or lactones preferably include organic carboxylic acids having at least one hydroxyl group in the molecules and having a molecular weight of between about 50 to about 1000. The molecular complex thus formed is optimally bioavailable for topical treatment of skin and nail diseases.

2. Description of Related Art

Transdermal delivery systems are a convenient and effective alternative for the administration of many types of medications, because the agents are delivered directly into the blood stream, avoiding first-pass metabolism in the liver, so that drug delivery is continuous and sustained. Transdermal delivery also provides a sustained and consistent delivery of medication, avoiding peaks and valleys in blood levels which are often associated with oral dosage forms. Thus, using transdermal delivery, one can administer lower doses of drug to achieve the same therapeutic effect compared to oral administration, reducing or eliminating dose-dependent side effects.

Preparing suitable formulations of medications is a challenging task, The skin, which has protective layers designed to prevent penetration of foreign matter, must be sufficiently penetrated to provide the active agent to the desired site or for absorption into the bloodstream. Skin is a complex organ system, consisting of multiple layers. The uppermost, or “stratum corneum,” layer of skin consists of non-living material derived primarily from the terminal differentiation of epidermal keratinocytes, and provides a protective barrier for the underlying components of skin. The epidermis contains a number of cell types, although keratinocytes are the major cell type. Dermal fibroblasts are embedded within a matrix comprised of collagen, elastin, proteoglycans, and other extracellular matrix molecules. Blood capillaries are found in the dermis, but the epidermis is non-vascular.

In addition, the drug itself must be suitable for administration. The size of a drug molecule, its charge, polarity, and pH are factors that contribute to the ability of the agent to penetrate the skin to the desired site or to blood vessels for systemic distribution. The carrier enabling the transdermal delivery of the drug has similar constraints.

Most transdermal delivery of pharmaceuticals involves incorporating the pharmaceutical into a carrier, such as a porous polymeric membrane, and using the membrane as a patch worn on the skin. Transdermal patch devices which provide a controlled, continuous administration of a therapeutic agent through the skin are known as the art. Such devices, for example, are disclosed in U.S. Pat. Nos. 4,627,429; 4,784,857; 5,662,925; 5,788,983; and 6,113,940. These devices typically contain a therapeutic agent impermeable barrier layer that defines the outer surface of the device, and a permeable skin attaching membrane, such as an adhesive layer, sealed to the barrier layer in such a way as to create a reservoir between them in which the therapeutic agent is placed. Although such devices may be satisfactory for their intended purpose, they have been found to be irritating to the wearer of the patch, provide minimized control of drug delivery through the skin, are slower to prepare, do not allow for customized formulation, are not easily produced, and are not cost-effective.

Numerous chemical agents have been studied as a means of increasing the rate at which a drug penetrates through the skin. As will be appreciated by those skilled in the art, chemical enhancers are compounds that are administered along with the drug (or in some cases the skin may be pretreated with a chemical enhancer) in order to increase the permeability of the stratum corneum, and thereby provide for enhanced penetration of the drug through the skin. Ideally, such chemical penetration enhancers are compounds that are innocuous and serve merely to facilitate diffusion of the drug through the stratum corneum. The permeability of many therapeutic agents with diverse physicochemical characteristics may be enhanced using these chemical enhancement means. However, there are skin irritation and sensitization problems associated with high levels of certain enhancers.

Many medicinal active agents contain one or more basic nitrogen atoms in their molecule and can therefore be utilized in pharmaceutical preparations either as a free base or as a salt of the active substance base with an acid which is suitable for this purpose. Salts have the advantage of better water solubility, which is important for oral administration, and in many cases also the advantage of better stability. A further advantage is that active substance salts often are more easily crystallized, or it is anyway only the active substance salt which is crystalline at room temperature. This is the reason why many active substances are manufactured and available only in the form of their salts. For example, chlorhexidine is commonly used as a salt of dihydrochloride, diacetate and di-D-gluconate. Erythromycin is commonly used as a salt of ethylsuccinate, acistrate, estolate, glucoheptonate, lactobionate, propionate and stearate.

For transdermal administration, however, the active substance salts are unsuitable since due to their higher polarity they are not capable of penetrating the lipophile barrier of the stratum corneum in the quantities required for the therapeutic purpose. Thus, it is necessary to transform active substance salts into their free base in order to utilize them in transdermal systems. Processes of making a topical composition comprising molecular complexes of these drugs with other vehicles for optimal bioavailability and improved delivery into the cutaneous tissues has not previously been described.

An ideal process enables the release of the free base during the manufacture of the system in situ without the manufacturing process thereby becoming considerably more complicated than in the case of direct use of the free base. Such a process is described in EP 0 272 562. In this process, adhesives are used which themselves possess basic groups and are thereby themselves, as auxiliary bases, capable of liberating the free base. The disadvantage of this process is that the number of these functional basic groups in the adhesive is limited, and that for this reason only small amounts of active substance salts can be converted into their free bases.

Another process is described in U.S. Pat. No. 6,620,429 where active substance salt is converted with a basic alkaline metal salt, preferably a silicate, in an organic solvent. The transdermal systems described therein involve incorporation of the converted active substance into a polymer matrix patch after suspension in the organic solvent with the basic alkaline metal salt. There is a need to develop a more convenient approach to transdermal drug delivery, so that the active drug becomes more readily available and easily transportable through cutaneous tissue.

U.S. Pat. No. 5,877,212, the disclosure of which is incorporated by reference herein in its entirety, discloses molecular complexes and sustained release formulations containing complexes formed between alpha hydroxyacids and related acids on the one hand, and a complexing agent on the other hand. The complexing agents include organic amino compounds in free base form having one or more other functional groups with unshared electrons such as hydroxyl, carbonyl, amido, ester, and alkoxy groups. The molecular complex provides for controlled release of the alpha hydroxyacid or related acid into the skin.

The description herein of certain disadvantages of known materials, methods, systems, and apparatus is not intended to limit the scope of the invention. Indeed, various embodiments of the invention may include some or all of the known materials, methods, systems, and apparatus without suffering from the aforementioned disadvantages.

### SUMMARY OF THE INVENTION

It is a feature of an embodiment of the invention to provide improved compositions and delivery systems to administer alkaline pharmaceutical drugs through the skin. It also a feature of an embodiment of the invention to provide methods of making the compositions, as well as methods of administering the compositions to a patient in need thereof.

In accordance with these and other features of various embodiments of the invention, there is provided a topical composition including a molecular complex formed between an alkaline pharmaceutical drug and at least one compound selected from a hydroxyacid, a polyhydroxy acid, a related acid, lactone forms of these acids, or combinations thereof.

In accordance with additional features of embodiments of the invention, there is provided a method of forming a molecular complex between an alkaline pharmaceutical drug and at least one of a hydroxyacid, polyhydroxyacid, related acid, and lactone. The method involves dissolving the alkaline pharmaceutical drug salt and an alkali in an appropriate medium to form a free base of the pharmaceutical drug, and then separating the free base from the medium. The method further includes adding at least one of a hydroxyacid, polyhydroxyacid, related acid, and lactone to the free base in a reaction medium to form a molecular complex.

In accordance with an additional feature of an embodiment of the invention, there is provided a method of administering an alkaline pharmaceutical drug to a patient in need thereof, comprising topically applying a molecular complex formed between an alkaline pharmaceutical drug and at least one compound selected from a hydroxyacid, a polyhydroxy acid, related acid, a lactone, or combinations thereof. The molecular complex includes a therapeutically effective amount of the alkaline pharmaceutical drug.

### DETAILED DESCRIPTION OF PREFERRED EMBODIMENTS

Embodiments of the invention are not limited to the particular methodology, protocols, and reagents described in the preferred embodiments, as these may vary. It also is to be understood that the terminology used herein is for the purpose of describing particular embodiments only, and is not intended to limit the scope of any embodiment of the present invention.

Throughout this disclosure, the singular forms “a,” “an,” and “the” include plural reference unless the context clearly dictates otherwise. Thus, for example, a reference to “an alkaline pharmaceutical drug” includes a plurality of such drugs, and a reference to “a hydroxacid” is a reference to one or more hydroxyacids and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are cited for the purpose of describing and disclosing the various molecules, drugs, delivery systems, and methodologies that are reported in the publications and that might be used in connection with the invention. Nothing herein is to be construed as an admission that the invention is not entitled to antedate such disclosures by virtue of prior invention.

The expression “pharmaceutically effective amount” is used herein to denote a quantity of pharmaceutical that is known to be effective to achieve the desired and known result of the drug. The actual amount contained in the molecular complex, likely will vary from the pharmaceutically effective amount, since some of the drug may not completely penetrate the skin together with the complex. Using the guidelines provided herein, those skilled in the art are capable of determining the pharmaceutically acceptable amount of alkaline pharmaceutical drugs described herein, and to use the requisite amount in the molecular complex so that the pharmaceutically acceptable amount is delivered to the subject in need thereof.

The expression “related acid” as it is used herein denotes a hydroxyacid in which the hydroxyl group is at any carbon position other than the alpha position, or the hydroxyl group is replaced by a keto group, or other miscellaneous organic hydroxycarboxylic acids that are not readily represented by a generic structure. For convenience this group of compounds may be subdivided into (1) alpha ketoacids, (2) miscellaneous compounds, and (3) oligomers and polymers of hydroxyacids. These groups are set out in more detail below.

In human skin, the stratum corneum consists of keratin-enriched corneocytes that are embedded in a lipid matrix and are resistant to penetration by ionic compounds or large molecules having a molecular weight of 800 or larger. Most alkaline pharmaceutical drugs are available in the form of a salt with inorganic acids such as hydrochloric acid, sulfuric acid and nitric acid because the free base is chemically unstable due to air oxidation of the amino, imino and/or guanido group of the molecule, and these drugs when oxidized typically become discolored and topically unappealing. When such inorganic salts are incorporated into a topical formulation, the pharmaceutical drug usually exists as a positively charged cation and cannot penetrate, or only partially penetrates the stratum corneum of the skin. The reason is believed to be due to the fact that the inorganic acids used for stabilization and isolation of the drug are strong acids and the drug molecule is fully ionized by such strong acids. The drug as a fully ionized cation is not in bioavailable form, and its topical effect is variable and inconsistent at best, and often is completely ineffective.

The present inventors have discovered a relatively simple process for converting the inorganic salt of an alkaline pharmaceutical drug into a molecular complex that provides the requisite bioavailability and therapeutic efficacy. In accordance with a preferred embodiment of the method, an inorganic salt of an alkaline pharmaceutical drug is reacted with equimolar amounts of an inorganic alkali such as sodium hydroxide or ammonium hydroxide to generate the free base of the drug. The free base of the drug then is reacted with an organic hydroxyacid, polyhydroxy acid, related acid, lactone, or combinations thereof, to form a molecular complex.

The expression “molecular complex” as used throughout this description to define the formation of a molecular complex between an alkaline pharmaceutical drug and the hydroxyacid, or polyhydroxy acid, related acid, or lactone denotes a complex based on three attracting forces. These three attracting forces in increasing strength are: (a) dipolar/dipolar; (b) dipolar/ionic; and (c) ionic/ionic. The dipolar attracting forces are created between the hydroxyl groups of: (i) the hydroxyacid or polyhydroxy acid or related acid, or lactone; and (ii) the amino, imino and/or guanido group of an alkaline drug due to unshared electrons of the oxygen and nitrogen atoms, and the hydrogen atoms through hydrogen bonds. The ionic attracting forces are created between the carboxyl group of the hydroxyacid or polyhydroxy acid, or related acid, or lactone on the one hand, and the protonated amino, imino or guanido group of an alkaline drug on the other hand.

When a composition containing the above molecular complex is topically applied to the skin, the drug molecules having a dipolar/dipolar attracting force will penetrate the skin first, followed by the drug molecules having dipolar/ionic attracting forces. The drug molecules having the ionic/ionic attracting forces are typically in salt form and therefore are not generally bioavailable for penetration into the skin. However, when more free base drug penetrates into the skin, the ionic drug molecule will become non-ionic because the cation is converted to a free base due to the dissociation equilibrium shift (Henderson-Hasselbalch Equation). Thus, most drug molecules become bioavailable in the molecular complex with a hydroxyacid or polyhydroxy acid, or related acid, or lactone.

The molar ratio of an alkaline drug to a hydroxyacid or polyhydroxy acid or related acid or lactone preferably ranges from about 1:0.1 to about 1:40, with a preferred range of from about 1:0.5 to about 1:5. The formation of a molecular complex is more than or beyond the neutralization reaction between an alkali and an acid because the extra functional group(s), e.g., hydroxyl group(s), participate in the formation of molecular complex through intermolecular attracting forces. The inventors believe that all alkaline pharmaceutical drugs that have amino, imino and/or quanido groups can form a molecular complex with hydroxyacids or polyhydroxy acids or related acids to provide a compound with improved bioavailability and improved delivery into the skin and nail plate.

The expression “alkaline pharmaceutical drug” denotes a pharmaceutical agent that is alkaline in its native form, but typically administered in its salt form, and that has a pharmaceutical effect. Representative alkaline pharmaceutical drugs include but are not limited to acebutolol, acetohydroxamic acid, actiq, acyclovir, albuterol, allopurinol, alloxanthine, alprazolam, alprenolol, amiloride, amantadine, aminacrine, amiodarone, amitriptyline, amorolfine, amodiaquin, amocarzine, amoxapine, amphetamine, atenolol, atropine, bemegride, benzocaine, bepridil, benztropine, bupivacaine, bupropion, burimamide, brompheniramine, butoconazole, caffeine, carbamazepine, chlordiazepoxide, chloroquine, chlorpheniramine, chlorpromazine, cimetidine, clonidine, cocaine, codeine, cyclizine, chlorhexidine, citalopram, clemastine, clindamycin, clioquinol, clotrimazole, clozapine, cromolyn, crotamiton, cyclizine, cycloserine, dexmedetomidine, dicyclomine, dihydromorphine, diphenhydramine, diphenoxylate, disopyramide, dobutamine, dopamine, dopamide, dopa esters, doxepin, doxylamine, dyclonine, desipramine, diazepam, dihydrocodeine, diphenoxylate, ephedrine, epinephrine, epinine, ergotamine, econazole, erythromycin, etidocaine, etomidate, fentanyl, fluoxetine, fluphenazine, flurazepam, fluvoxamine, guanethidine, guaifenesin, N-guanylhistamine, haloprogin, hydralazine, hypoxanthine, ichthammol, imiquimod, indomethacin, imipramine, irbesartan, isoetharine, isoproterenol, ketamine, ketanserin, ketoconazole, ketoprofen, kanamycin, labetalol, lamotrigine, lidocaine, lobeline, losartan, loxapine, lysergic diethylamide, mafenide, maprotiline, mecamylamine, meclizine, meclocycline, meperidine, mepivacaine, mescaline, metanephrine, metaproterenol, methadone, methoxamine, metiamide, metolazone, metronidazole, miconazole, midazolam, minocycline, minoxidil, mirtazapine, mupirocin metaraminol, methadone, methamphetamine, methyldopamide, methyldopa esters, metoprolol, mexiletine, molindone, morphine, moxonidine, 3,4-methylenedioxymethamphetamine, nadolol, naftifine, naloxone, nefazodone, neomycin, nifedipine, nystatin, nicotine, norepinephrine, octopamine, olanzapine, ondansetron, oxiconazole, oxotremorine, oxymetazoline, paroxetine, pentazocine, phencyclidine, pheniramine, phenmetrazine, phentolamine, phenylephrine, phenylpropanolamine, phenelzine, phenoxybenzamine, physostigmine, pilocarpine, pimozide, pipamazine, pirenzepine, podophyllin, podofilox, pramipexole, pramoxine, prenalterol, prilocalne, procaine, promethazine propionate, propranolol, protriptyline, pseudoephedrine, pyrethrin, pyrilamine pentazocine, phenylephrine, physostigmine, pilocarpine, pindolol, prazosin, procainamide, procaine, promazine, promethazine, propranolol, pseudoephedrine, pyrimethamine, quetiapine, quinethazone, quinidine, reserpine, risperidone, ritodrine, ropinirole, ropivacaine, salmeterol, scopolamine, selegiline, serotonin, sertindole, sertraline, sotalol, strychnine, sulconazole, sulfadiazine, sulfanilamide, tamsulosin, tazarotene, terbinafine, terconazole, terfenadine, tetracaine, tetracycline, tetrahydrozoline, theobromine, theophylline, thymol, timolol, tioconazole, tizanidine, tocamide, tolnaftate, tranylcypromine, trazodone, triamterene, triazolam, triflupromazine, tripelennamine, triprolidine, terbutaline, thioridazine, tyramine, tolazoline, xanthine, venlafaxine, verapamil and ziprasidone, and mixtures thereof.

The hydroxyacids and polyhydroxy acids useful in forming a molecular complex with the alkaline pharmaceutical drugs mentioned previously are described in more detail below. Suitable hydroxyacids may be divided into the following groups.

1. Alpha-Hydroxyacids (AHAs)

AHAs are organic carboxylic acids having one hydroxyl group attached directly to the alpha position of the aliphatic or alicyclic carbon atom, but not to a benzene or other aromatic ring. On a broader scope, AHAs may include those acids that have additional carboxyl groups. The AHAs may be divided into three subgroups: (a) alkyl AHAs; (b) aralkyl AHAs; and (c) polycarboxyl AHAs.
(a) Alkyl AHAs

The side chain radicals attached to the alpha carbon are hydrogen atoms or simple hydrocarbons called alkyl groups. The generic structure may be represented as follows:
R₁R₂C(OH)COOH
where R₁ and R₂ may be independently H or alkyl group. The alkyl AHAs may exist as stereoisomers as D, L and DL or R, S and RS forms when R₁ and R₂ are not identical. The alkyl groups preferably are non-aromatic radicals such as methyl, ethyl, propyl, isopropyl, butyl, pentyl, octyl, lauryl and stearyl.

Representative alkyl AHAs can be selected from the group consisting of 2-hydroxyethanoic acid (glycolic acid), 2-hydroxypropanoic acid (lactic acid), 2-methyl-2-hydroxypropanoic acid (methyl)acetic acid), 2-hydroxybutanoic acid, 2-hydroxypentanoic acid, 2-hydroxyhexanoic acid, 2-hydroxyheptanoic acid, 2-hydroxyoctanoic acid, 2-hydroxyeicosanoic acid (alpha hydroxyarachidonic acid), 2-hydroxytetraeicosanoic acid (cerebronic acid), 2-hydroxytetraeicosenoic acid (alpha hydroxynervonic acid), and mixtures thereof.
(b) Aralkyl AHAs

Aralkyl AHAs include AHA having aralkyl groups, where aralkyl is an abbreviation for aryl plus alkyl. An aralkyl AHA is formed when a phenyl group or other aromatic ring is attached to the alpha carbon of the alkyl AHA. The generic structure is shown as follows.
R₁R₂C(OH)COOH
where R₁ and R₂ may be independently H, aryl or aralkyl group. The aralkyl AHAs may exist as stereoisomers as D, L and DL or R, S and RS forms when R₁ and R₂ are not identical. The aryl group preferably includes at least one aromatic radical such as phenyl, diphenyl, biphenyl and naphthyl. The aralkyl group preferably includes at least one aromatic radical and one non-aromatic radical such as a phenylmethyl (benzyl), phenylethyl, phenylpropyl, diphenylmethyl, diphenylethyl, biphenylmethyl and naphthylmethyl group. In any case, the hydroxyl group is attached to the non-aromatic alpha carbon atom.

Suitable aralkyl AHAs can be selected from the group 2-henyl-2-hydroxyethanoic acid (mandelic acid), 2,2-diphenyl-2-hydroxyethanoic acid (benzilic acid), 3-phenyl 2-hydroxypropanoic acid (3-phenyllacetic acid), 2-phenyl-2-methyl-2-hydroxyethanoic acid (atrolactic acid, 2-phenyllacetic acid), and mixtures thereof.
(c) Polycarboxy AHAs

A polycarboxy AHA is an AHA that includes more than one carboxyl and/or hydroxyl group. The generic structure may be shown as follows.
R₁R₂C(OH)COOH
where R₁ and R₂ may be independently H, COOH, CH₂COOH or CHOHCOOH. Suitable polycarboxy AHAs may exist as stereoisomers as D, L and DL or R, S and RS forms when R₁ and R₂ are not identical.

Suitable polycarboxy AHAs can be selected from the group 2-hydroxypropane-1,3-dioic acid (tartronic acid), 2-hydroxybutane-1,4-dioic acid (malic acid), 2,3-dihydroxybutane-1,4-dioic acid (tartaric acid), 2-hydroxy-2-carboxypentane-1,5-dioic acid (citric acid), isocitric acid, and mixtures thereof.

2. Beta-Hydroxyacids (BHAs)

BHAs are organic carboxylic acids having one hydroxyl group attached to the beta position of aliphatic carbon atom. The generic structure of a BHA typically is represented by the following formula:
R₁R₂C(OH)CHR₃COOH
where R₁, R₂, R₃ may be H, alkyl, aryl or aralkyl group. The BHA may exist as stereoisomers as D, L and DL or R, S and RS forms when R₁ and R₂ are not identical or R₃ is not H.

Suitable BHAs for use in the present invention can be selected from the group 3-hydroxypropanoic acid (β-hydroxypropanoic acid), 3-hydroxybutanoic acid (β-hydroxybutanoic acid), 3-hydroxypentanoic acid, 3-hydroxy-2-phenylpropanoic acid (tropic acid), and mixtures and combinations thereof. For clarification, salicylic acid is not a BHA because both the hydroxyl and carboxyl groups are attached directly to an aromatic benzene ring, and the chemical name is 2-hydroxybenzoic acid.

3. Polyhydroxy Acids (PHAs)

PHAs are organic carboxylic acids having multiple hydroxyl groups in addition to the alpha-hydroxyl group. The PHAs typically exist in the lactone form, such as gluconolactone from gluconic acid. Many PHAs are derived from carbohydrates and are important carbohydrate intermediates and metabolites. PHAs may be divided into three groups: (a) aldonic acid; (b) aldaric acid; and (c) alduronic acid.
(a) Aldonic Acid

When a common carbohydrate such as glucose, also called aldose, is oxidized at carbon one position from aldehyde to carboxyl group, the product is called aldonic acid, or more specifically gluconic acid. The aldonic acid usually has multiple hydroxyl groups. The generic structure for aldonic acids is provided by the following formula.
R(CHOH)ₙCHOHCOOH
where R is usually H or alkyl group; and n is an integer from 1-6. The aldonic acids may exist as stereoisomers as D, L and DL, or R, S and RS forms. Many aldonic acids form intramolecular lactones by the removal of one mole of water between the carboxyl group and one hydroxyl group.

Representative aldonic acids can be selected from the group 2,3-dihydroxypropanoic acid (glyceric acid), 2,3,4-trihydroxybutanoic acids (stereoisomers; erythronic acid and erythronolactone, threonic acid and threonolactone), 2,3,4,5-tetrahydroxypentanoic acids (stereoisomers; ribonic acid and ribonolactone, arabinoic acid and arabinolactone, xylonic acid and xylonolactone, lyxonic acid and lyxonolactone), 2,3,4,5,6-pentahydroxyhexanoic acids (stereoisomers; allonic acid and allonolactone, altronic acid and altronolactone, gluconic acid and gluconolactone, mannoic acid and mannolactone, gulonic acid and gulonolactone, idonic acid and idonolactone, galactonic acid and galactonolactone, talonic acid and talonolactone), 2,3,4,5,6,7-hexahydroxyheptanoic acids (stereoisomers; alloheptonic acid and alloheptonolactone, altroheptonic acid and altroheptonolactone, glucoheptonic acid and glucoheptonolactone, mannoheptonic acid and mannoheptonolactone, guloheptonic acid and guloheptonolactone, idoheptonic acid and idoheptonolactone, galactoheptonic acid and galactoheptonolactone, taloheptonic acid and taloheptonolactone), and mixtures thereof.
(b) Aldaric Acid

Aldaric acid typically has multiple hydroxyl groups attached to the carbon chain surrounded by two carboxyl groups. Many aldaric acids exist as lactones, such as glucarolactone. The generic structure is represented by the following formula:
HOOC(CHOH)ₙCHOHCOOH
where n is an integer from 1-4. The aldaric acids may exist as stereoisomers as D, L and DL, or R, S and RS forms. Many aldaric acids form intramolecular lactones by the removal of one mole of water between one carboxyl group and one hydroxyl group.

Representative aldaric acids can be selected from the group consisting of 2,3-dihydroxybutane-1,4-dioic acids (stereoisomers; erythraric acid and threaric acid, also known as tartaric acid), 2,3,4-trihydroxypentane-1,5-dioic acids (stereoisomers; ribaric acid and ribarolactone, arabaric acid and arabarolactone, xylaric acid and xylarolactone, lyxaric acid and lyxarolactone), 2,3,4,5-tetrahydroxyhexane-1,6-dioic acids (stereoisomers; allaric acid and allarolactone, altraric acid and altrarolactone, glucaric acid and glucarolactone, mannaric acid and mannarolactone, gularic acid and gularolactone, idaric acid and idarolactone, galactaric acid and galactarolactone, talaric acid and talarolactone), 2,3,4,5,6-pentahydroxyheptane-1,7-dioic acids (stereoisomers; alloheptaric acid and alloheptarolactone, altroheptaric acid and altroheptarolactone, glucoheptaric acid and glucoheptarolactone, mannoheptaric acid and mannoheptarolactone, guloheptaric acid and guloheptarolactone, idoheptaric acid and idoheptarolactone, galactoheptaric acid and galactoheptarolactone, taloheptaric acid and taloheptarolactone), and mixtures thereof.
(c) Alduronic Acid

Alduronic acid preferably is obtained from a carbohydrate, aldose, by oxidation of the terminal carbon to a carboxyl group, and the carbon one position remains as an aldehyde group, such as glucuronic acid from glucose. Similar to aldonic acid and aldaric acid, alduronic acid also has multiple hydroxyl groups attached to the carbon chain between two functional groups, one aldehyde and one carboxyl groups in this case. Many alduronic acids exist as lactones, such as glucuronolactone from glucuronic acid. The generic structure is represented by the following formula:
HOOC(CHOH)ₙCHOHCHO
where n is an integer from 1-4. The alduronic acids may exist as stereoisomers as D, L and DL, or R, S and RS forms. Many alduronic acids can form intramolecular lactones by the removal of one mole of water between the carboxyl group and one hydroxyl group.

Representative alduronic acids can be selected from the group consisting of erythruronic acid, threuronic acid, riburonic acid and riburonolactone, araburonic acid and araburonolactone, xyluronic acid and xyluronolactone, lyxuronic acid and lyxuronolactone, alluronic acid and alluronolactone, altruronic acid and altruronolactone, glucuronic acid and glucuronolactone, mannuronic acid and mannuronolactone, guluronic acid and guluronolactone, iduronic acid and iduronolactone, galacturonic acid and galacturonolactone, taluronic acid and taluronolactone, allohepturonic acid and allohepturonolactone, altrohepturonic acid and altrohepturonolactone, glucohepturonic acid and glucohepturonolactone, mannohepturonic acid and mannohepturonolactone, gulohepturonic acid and gulohepturonolactone, idohepturonic acid and idohepturonolactone, galactohepturonic acid and galactohepturonolactone, talohepturonic acid and talohepturonolactone, and mixtures thereof.

4. Aldobionic Acids (ABAs)

ABAs are also known as bionic acids, and typically consist of one monosaccharide chemically linked through an ether bond to an aldonic acid. The ABA also may be described as an oxidized form of a disaccharide or dimeric carbohydrate, such as lactobionic acid from lactose. In most ABAs, the carbon at position one of the monosaccharide is chemically linked to a hydroxyl group at different position of the aldonic acid. Therefore, different ABAs or stereoisomers can be formed from two identical monosaccharides and aldonic acids. Similar to PHAs, ABAs have multiple hydroxyl groups attached to carbon chains. ABAs may be represented by the following generic formula:
H(CHOH)ₘ(CHOR)(CHOH)ₙCOOH
where m and n are integers independently from 0-7, and R is a monosaccharide. ABAs may exist as stereoisomers as D, L and DL, or R, S and RS forms, and can form intramolecular lactones by the removal of one mole of water between the carboxyl group and one hydroxyl group. Chemical structures of most ABAs are more complicated than the above generic formula. Accordingly, the ABAs useful in forming the molecular complex of the invention will be described by reference to their chemical names.

Suitable ABAs useful in embodiments of the invention may be selected from the group consisting of lactobionic acid and lactobionolactone from lactose, isolactobionic acid and isolactobionolactone from isolactose, maltobionic acid and maltobionolactone from maltose, isomaltobionic acid and isomaltobionolactone from isomaltose, cellobionic acid and cellobionolactone from cellobiose, gentiobionic acid and gentiobionolactone from gentiobiose, kojibionic acid and kojibionolactone from kojibiose, laminaribionic acid and laminaribionolactone from laminaribiose, melibionic acid and melibionolactone from melibiose, nigerobionic acid and nigerobionolactone from nigerose, rutinobionic acid and rutinobionolactone from rutinose, sophorobionic acid and sophorobionolactone from sophorose, and mixtures thereof.

Preferred hydroxacids, polyhydroxyacids, and lactones, or combinations thereof, include glycolic acid, lactic acid, gluconic acid, gluconolactone, ribonic acid, ribonolactone, galactonic acid, galactonolactone, glucoheptonic acid, glucoheptonolactone, glucuronic acid, glucuronolactone, galacturonic acid, galacturonolactone, glucaric acid, glucarolactone, galactaric acid, galactarolactone, lactobionic acid and maltobionic acid.

5. Related Acids

The related acids are those hydroxyacids in which the hydroxyl group is at any carbon position other than the alpha position, or the hydroxyl group is replaced by a keto group, or other miscellaneous organic hydroxycarboxylic acids which are not readily represented by a generic structure. For convenience this group of compounds is subdivided into (1) alpha ketoacids, (2) miscellaneous compounds, and (3) oligomers and polymers of hydroxyacids.
(a) Alpha Ketoacids

Ketoacids are related to hydroxyacids in that the hydroxyl group is replaced by the keto group. Although the keto group can be at any position other than the terminal ends, the preferred one is an alpha ketoacid. For example pyruvic acid, an alpha ketoacid is related to lactic acid in that the hydroxyl group of lactic acid is substituted by a keto group. In the skin, lactate dehydrogenase enzyme converts pyruvate to lactate and vice visa. The ketoacids have been found to have similar therapeutic effects as that of alpha hydroxyacids. The generic structure of alpha ketoacids may be represented as follows:
(Ra)COCOOH
wherein Ra is H, alkyl, aralkyl or aryl group of saturated or unsaturated, isomeric or non-isomeric, straight or branched chain or cyclic form, having 1 to 25 carbon atoms, and in addition Ra may carry F, Cl, Br, I, OH, CHO, COOH and alkoxyl group having 1 to 9 carbon atoms. The typical alkyl, aralkyl, aryl and alkoxyl groups for Ra include methyl, ethyl, propyl, isopropyl, butyl, pentyl, octyl, lauryl, stearyl, benzyl, phenyl, methoxyl and ethoxyl.

Representative alpha ketoacids that may be useful for forming the molecular complex of the invention are listed below: 2-ketoethanoic acid (glyoxylic acid), 2-ketopropanoic acid (pyruvic acid), 2-phenyl-2-ketoethanoic acid (benzoylformic acid), 3-phenyl-2-ketopropanoic acid (phenylpyruvic acid), 2-ketobutanoic acid, 2-ketopentanoic acid, 2-ketohexanoic acid, 2-ketoheptanoic acid, 2-ketooctanoic acid and 2-ketododecanoic acid.
(b) Miscellaneous Hydroxyacids

These hydroxyacids have similar therapeutic effects as that of alpha hydroxyacids but their chemical structures are not readily represented by the foregoing generic structures. These compounds are listed as follows: agaricic acid, aleuritic acid, citramalic acid, glucosaminic acid, galactosaminic acid, 2-keto-gulonic acid and 2-keto-gulonolactone, mannosaminic acid, mevalonic acid and mevalonolactone, pantoic acid and pantolactone, quinic acid (1,3,4,5-tetrahydroxycyclohexanecarboxylic acid), piscidic acid (4-hydroxybenzyltartaric acid), ascorbic acid (3-oxo-L-gulofuranolactone), Isoascorbic acid (D-erythro-hex-2-enonic acid-lactone), 2-hexylosonic acids (isomers; arabino-2-hexylosonicacid, xylo-2-hexylosonic acid, ribo-2-hexylosonic acid, lyxo-2-hexylosonic acid), 5-hexylosonic acids (isomers; arabino-5-hexylosonic acid, xylo-5-hexylosonic acid, ribo-5-hexylosonic acid, lyxo-5-hexylosonic acid).
(c) Oligomers of Hydroxyacids

When two or more molecules of hydroxyacids either identical or non-identical are reacted chemically to each other, oligomers are formed. The chemical bond is usually an ester bond formed from the carboxyl group of one monomer and the hydroxyl group of a second monomer by eliminating a water molecule. In general, oligomers consist of 2 to 10 monomers of hydroxyacids. The oligomers may be cyclic or non-cyclic form or a mixture of the two. The generic structure of oligomers of hydroxyacids may be described as follows.
(AHA)ₘ-n(H₂O)
wherein, AHA is a hydroxyacid described above, m=2-10, with a preferred number of 2-4, and n=m−1. AHA in each monomer may be identical or not identical. For example, glycolyl glycolate, glycolyl lactate, lactyl lactate and lactyl glycolate. Representative oligomers of AHA are listed below: glycolyl glycolate, lactyl lactate, citryl citrate, glycoly citrate, citryl glycolate, lactyl citrate, citryl lactate, malyl malate, malyl glycolate, tartaryl tartrate, tartaryl glycolate, glycolyl tartrate, glycolyl glycoly glycolate, lactyl lactyl lactate, and other AHA oligomers. It is preferred that the molecular weight of the polymeric hydroxyacid be within the range of from about 50 to about 1000.

Because the molecular complex should be effective in permitting the release of the drug through the skin, it is preferred that the molecular weight of the hydroxyacid, or polyhydroxyacid, or related acid, or lactone form thereof be within the range of from about 50 to about 1000. It is more preferred that the molecular weight be within the range of from about 60 to about 700, and most preferred within the range of from about 70 to about 500.

The molecular complex formed from an alkaline drug and a hydroxyacid or polyhydroxy acid has been found to provide optimal bioavailability for topical treatment of various dermatological indications. A therapeutic molecular complex can also be formed between an alkaline drug and N-acetylamino acid. Typical N-acetylamino acids are described in U.S. Pat. No. 6,159,485, the disclosure of which is incorporated by reference herein in its entirety. Representative N-acetylamino acids include N-acetyl-L-proline, N-acetyl-L-glutamine, N-acetyl-L-cysteine and N-acetyl-glycine.

The beneficial effects of forming the molecular complexes of various embodiments of the invention are readily apparent when considering, for example, that a 2% miconazole nitrate formulation is not therapeutically effective for topical treatment of fungal infections of nails. In contrast, a 1-2% miconazole molecular complex with glycolic acid has been found to be therapeutically effective for the topical treatment of nails with fungal infections. In a similar fashion, diphenhydramine hydrochloride (2% formulation) is not therapeutically effective for topical treatment of skin itch, but a 2% diphenhydramine molecular complex with gluconolactone has been found to be topically effective for eradication of skin itch. These unexpected and surprising results can be realized with molecular complexes of other alkaline pharmaceutical drugs.

The molecular complex composition also may preferably contain other pharmaceutical or topical agents to further expand the utilities for maximal therapeutic efficacies, such as in combination with N-acetylamino sugars as disclosed in U.S. Pat. No. 6,159,485, the disclosure of which is incorporated by reference herein in its entirety. Suitable pharmaceutical and other topical agents that may be incorporated into embodiments of the molecular complex compositions of the invention include: those that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; antiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants; hormones; retinoids; gum disease or oral care agents; topical cardiovascular agents; corn, callus and wart removing agents; dipilating agents, and mixtures and combinations thereof.

Other useful pharmaceutical and other topical agents that can be included in embodiments of the molecular complex compositions of the invention include those selected from the group consisting of aclovate, acyclovir, acetylsalicylic acid, adapalene, aluminum acetate, aluminum chloride, aluminum hydroxide, aluminum chlorohydroxide, aminobenzoic acid (PABA), aminocaproic acid, aminosalicylic acid, anthralin, ascorbic acid, ascoryl palimate, azelaic acid, bacitracin, bemegride, beclomethasone dipropionate, benzophenone, benzoyl peroxide, betamethasone dipropionate, betamethasone valerate, calcipotriene, camphor, capsaicin, carbamide peroxide, chitosan, chloroxylenol, ciclopirox, clobetasol propionate, coal tar, dehydroepiandrosterone, desoximetasone, dexamethasone, estradiol, ethinyl estradiol, fluocinonide, fluocinolone acetonide, 5-fluorouracil, griseofulvin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, indomethacin, kojic acid, menthol, methyl nicotinate, methyl salicylate, monobenzone, naproxen, octyl methoxycinnamate, octyl salicylate, oxybenzone, padimate O, permethrin, phenol, piperonyl butoxide, povidone iodine, resorcinol, retinal, 13-cis retinoic acid, retinoic acid, retinol, retinyl acetate, retinyl palmitate, salicylamide, salicylic acid, selenium sulfide, shale tar, sulfur, triamcinolone diacetate, triamcinolone acetonide, triamcinolone hexacetonide, triclosan, undecylenic acid, urea, vitamin E acetate, wood tar, zinc pyrithione, N-acetyl-prolinamide, N-acetyl-lysine, N-acetyl-ornithine, N-acetyl-glucosamine, and mixtures thereof.

The present inventors also have discovered that compositions comprising a molecular complex of preferred embodiments of the present invention are topically effective for the general care of skin, hair and nail; nasal, oral and vaginal mucosa. The compositions are useful in a variety of methods, including: treatment, healing and prevention of cosmetic conditions and dermatological indications, as well as cosmetic and clinical signs of changes associated with intrinsic or extrinsic aging; the damages caused by extrinsic factors such as sunlight, air pollution, wind, cold, dampness, heat, chemicals, smoke, cigarette smoking, and radiations including electromagnetic radiations and ionizing radiations. The compositions also are useful for reducing and soothing mucosa and skin erythema, inflammation or reaction caused by internal or external factors.

General cosmetic conditions and dermatological indications that can be treated using the molecular complexes of various embodiments of the invention include: disturbed keratinization, inflammation, defective syntheses of dermal components, and changes associated with intrinsic and extrinsic aging of skin, nail and hair. Particular conditions and indications include: dryness or looseness of skin, nail and hair; xerosis; ichthyosis; palmar and plantar hyperkeratoses; uneven and rough surface of skin, nail and hair; dandruff; Darier's disease; lichen simplex chronicus; keratoses; acne; pseudofolliculitis barbae; dermatoses; eczema; psoriasis; pruritus; warts; herpes; age spots; lentigines; melasmas; blemished skin; hyperkeratoses; hyperpigmented or hypopigmented skin; abnormal or diminished syntheses of collagen, glycosaminoglycans, proteoglycans and elastin as well as diminished levels of such components in the dermis; stretch marks; skin lines; fine lines; wrinkles; thinning of skin, nail plate and hair; skin thickening due to elastosis of photoaging, loss or reduction of skin, nail and hair resiliency, elasticity and recoilability; lack of skin, nail and hair lubticants and luster; dull and older-looking skin, nail and hair; fragility and splitting of nail and hair, or used as to lighten the skin.

Specific skin changes associated with aging include, but are not limited to, progressive thinning of skin, fragile skin, deepening of skin lines and fine lines, wrinkles including fine and coarse wrinkles, lusterless skin surface, coarse and uneven skin, loss of skin elasticity and recoilability, blemished and leathery skin, loss of skin lubricating substances, increased numbers of blotches and mottles, nodules, pre-cancerous lesions, pigmented spots and mottled skin, changes in qualities and quantities of collagen and elastic fibers, solar elastosis, decrease in collagen fibers, diminution in the number and diameter of elastic fibers in the papillary dermis, atrophy of the dermis, stretch marks, reduction in subcutaneous adipose tissue and deposition of abnormal elastic materials in the upper dermis, yellowing skin, telangiectatic skin and older-looking skin.

A particularly preferred process for forming the molecular complex of the invention includes dissolving an alkaline pharmaceutical drug (0.1 mole in salt form) together with a sufficient amount of water (e.g., about 50 ml given the amount of drug). After dissolution, about 5N sodium hydroxide (20 ml) can be added slowly with stirring while the reaction flask is cooled externally in an ice-water bath. The free base of the drug is formed instantly and is usually separated as a precipitate or an oily product. The precipitate then can be isolated by filtration and washed with water and dried. The oily product can be isolated and washed with water using a separatory funnel.

To prepare a typical molecular complex composition, the above free base drug (0.1 mole) isolated as a precipitate or oily liquid then preferably is suspended in water (e.g., about 50 ml) and a hydroxyacid or polyhydroxy acid is added with stirring. Alternatively, other solvents such as ethanol, propylene glycol, butylene glycol, etc may be added to the water solution before or after the formation of the molecular complex. The formation of the molecular complex is evidenced by a decrease of the pH, and the reaction is completed as shown by no more change in the pH. The concentration of hydroxyacid or polyhydroxy acid or lactone may vary anywhere from about 0.1 to about 40 moles, preferably from about 0.5 to about 5 moles, per one mole of alkaline drug. The final pH of a composition containing a molecular complex may range from about 2.0 to about 7.0, with a preferred pH within the range of from about 3.0 to about 5.0.

To prepare a synergistic or synergetic composition, a pharmaceutical or other topical agent can be added directly or first dissolved in water or other solvent and then added into a composition containing a molecular complex of an embodiment of the invention. Other forms of compositions such as a solution, lotion, cream, ointment, gel etc. for topical delivery of the molecular complex containing an alkaline drug and a hydroxyacid or polyhydroxy acid or lactone of the instant invention can readily be prepared or formulated by those skilled in the art, using the guidelines provided herein.

The concentration of the alkaline pharmaceutical drug may range anywhere from 0.01 to 99.9%, with preferred concentration of from about 0.1 to 50% and with more preferred concentration of from about 1 to 25% by weight of the total composition. Other advantageous concentration ranges provide a concentration of at least 3%, 4% or 5% of the alkaline pharmaceutical drug. Higher concentrations of an alkaline pharmaceutical drug in the ranges of 40%, 50%, 60% or more also can be employed, depending on the desired end use. Thus, acceptable ranges of an alkaline pharmaceutical drug will be from about 1%, 2%, 3%, 4% or 5% at the minimum, to about 95% at maximum, and within that range will be ranges of from about 1% to about 5%, from about 5% to about 10%, from about 10% to about 20%, from about 20% to about 40%, from about 40% to about 60%, from about 60% to about 80%, from about 80% to about 95%. These weights are based on the weight of the total composition.

The concentration of the hydroxyacid, polyhydroxy acid, related acid, or lactone forms of these acids, or combinations thereof, (collectively referred to as “hydroxyacid” in this paragraph) may range from 0.01 to 99.9%. Advantageous concentrations will comprise at least 0.2% hydroxyacid, and typically at least about 1% or 2% of hydroxyacid. Other advantageous concentration ranges provide at least being at least 3%, 4% or 5% of a hydroxyacid. Higher concentrations of a hydroxyacid in the ranges of 40%, 50%, 60% or more also can be employed. Thus, typical ranges of a hydroxyacid will be from about 1%, 2%, 3%, 4% or 5% at the minimum to 99.9% at maximum, and within that range will be ranges of from about 5% to about 10%, from about 10% to about 20%, from about 20% to about 40%, from about 40% to about 60%, from about 60% to about 80%, from about 80% to about 99.9%. These weights are based on the weight of the total composition.

To prepare a topical composition in lotion, cream or ointment form, the above aqueous mixture containing the molecular complex preferably is mixed in a conventional manner with a commonly available lotion, cream or ointment base. A topical composition of the instant invention may also be formulated in a gel form. A typical gel composition can be prepared by the addition of a gelling agent such as methyl cellulose, ethyl cellulose, hydroxyethylcellulose, hydroxypropylcellulose, hydroxypropylmethylcellulose, carbomer or ammonium glycyrrhizate to a solution mixture containing the molecular complex. The preferred concentration of the gelling agent may range from 0.1 to 4 percent by weight of the total composition.

The following are illustrative examples of formulations and test results, and are not limiting. Therefore, any of the aforementioned alkaline drugs, hydroxyacids and polyhydroxy acids and lactones can be substituted according to the teachings of this invention in the following examples.

### Example 1

A typical process to convert a pharmaceutical drug from its salt form to a free base form is described as follows. Diphenhydramine hydrochloride 29 g (0.1 mole) was dissolved in water (50 ml) and 5N sodium hydroxide-(20 ml) was slowly added to generate diphenhydramine as a free base as shown by the formation of oily precipitates and the change from pH 5.5 to 9.4. Gluconolactone 18 g (0.1 mole) was added to form a molecular complex between the diphenhydramine free base and gluconic acid/gluconolactone as shown by the disappearance of the oily precipitates and the change from pH 9.4 to 7.4. The formation of the molecular complex was completed as indicated by no more change in pH of the solution. The solution thus obtained contained 0.1 mole diphenhydramine in molecular complex with 0.1 mole gluconic acid/gluconolactone. This concentrated stock solution was used for various forms of topical formulations including oil-in-water creams, lotions, gels and solutions.

### Example 2

An alternative method of forming the molecular complex is to use ammonium hydroxide instead of sodium hydroxide as follows. Diphenhydramine hydrochloride 29 g (0.1 mole) was dissolved in water 50 ml and concentrated ammonium hydroxide 6.9 ml (0.1 mole) was slowly added to generate diphenhydramine as a free base as shown by the formation of oily precipitates and the change from pH 5.5 to 8.0. Gluconolactone 18 g (0.1 mole) was added to form a molecular complex between diphenhydramine as a free base and gluconic acid/gluconolactone as shown by the disappearance of the oily precipitates and the change from pH 8.0 to 4.8. The formation of the molecular complex was completed as indicated by no more change in pH of the solution. The solution thus obtained contained 0.1 mole diphenhydramine in molecular complex with 0.1 mole gluconic acid/gluconolactone. This concentrated stock solution was used for various forms of topical formulations including creams, lotions, gels and solutions.

### Example 3

The molar ratio of the molecular complex may be changed from 1:1 to 1:2 by carrying out the following. Diphenhydramine hydrochloride 29 g (0.1 mole) was dissolved in water 50 ml and concentrated ammonium hydroxide 6.9 ml (0.1 mole) was slowly added to generate diphenhydramine as a free base as shown by the formation of oily precipitates and a change from pH 5.5 to 8.0. Gluconolactone 36 g (0.2 mole) then was added to form a molecular complex between the diphenhydramine free base and gluconic acid/gluconolactone as shown by the disappearance of the oily precipitates and a change from pH 8.0 to 3.2. The formation of molecular complex was completed as indicated by no more change in pH of the solution. The solution thus obtained contained 0.1 mole diphenhydramine in molecular complex with 0.2 mole gluconic acid/gluconolactone. This concentrated stock solution was used for various forms of topical formulations including solutions, lotions, creams and gels.

### Example 4

The molecular complex of diphenhydramine and gluconic acid/gluconolactone obtained from Example 1, 2, or 3 was mixed with an oil-in-water base to form a cream containing 2% of the active ingredient. A male subject, age 71, with chronic nummular eczema and pruritic dry skin topically applied the above 2% diphenhydramine cream containing molecular complex 1:1 or 1:2 ratio to itchy skin areas of eczema and dry skin lesions. A few minutes after the topical application, the itch disappeared completely and the lesions remained free of itch for the next 8 hours.

### Example 5

For alternative treatment of eczema and other dermatoses, hydrocortisone 17-valerate (0.2 g) first was dissolved in warm propylene glycol 20 ml, and the solution thus obtained was mixed with 79.8 g of molecular complex containing 2 g of diphenhydramine and 2.4 g of gluconic acid/gluconolactone in oil-in-water cream. The synergetic composition thus formulated contained 0.2% hydrocortisone 17-valerate, 2% diphenhydramine, and 2.4% gluconic acid/gluconolactone, and was therapeutically effective for topical treatment to eradicate itch and improve eczematous or psoriatic lesions.

### Example 6

Clotrimazole is commercially available as a free base powder, but it is chemically unstable in a solution or formulation for shelf storage due to air oxidation. A molecular complex composition can be formulated as follows.

Clotrimazole 2 g (5.8 mmole) was dissolved in 84 ml solution prepared from water (40 parts), ethanol (40 parts), and propylene glycol (20 parts), each part by volume. Glycolic acid, as a 70% aqueous solution, (14 ml-162.5 mmole) was added slowly to form a molecular complex as shown by a change of pH to 2.2. The molecular complex thus prepared contained 2% clotrimazole and 12% glycolic acid in solution form.

A male subject, age 64, having fungal infections on the left great toe nail for several months topically applied the above molecular complex once daily on the infected nail plate. After 8 months of topical treatment, there was no clinical signs of fungal infections and the nail grew to the normal length. This result reveals that the molecular complex formed between clotrimazole and glycolic acid is therapeutically effective for topical treatment of fungal infections.

### Example 7

Clotrimazole 2 g (5.8 mmole) was dissolved in 93 ml solution prepared from water (40 parts), ethanol (40 parts), and propylene glycol (20 parts), each part by volume. N-Acetyl-L-proline 5 g (32 mmole) was added slowly to form a molecular complex as indicated by a change of pH to 3.8. The solution thus obtained contained a molecular complex formed between 2% clotrimazole and 5% N-acetyl-L-proline that is useful for fungal infections of skin and nails.

### Example 8

Miconazole nitrate 47.9 g (0.1 mole) was suspended in water (50 ml), ethanol (50 ml), propylene glycol (50 ml), and 2N sodium hydroxide 50 ml (0.1 mole) was added with stirring. A sticky solid was initially formed from the mixture and became white crystals after continued stirring. The mixture was filtered and the white crystals were washed with water and dried. Miconazole free base, 42 g (0.1 mole) thus isolated, was used for the following preparation of a molecular complex.

Miconazole free base 8.2 g (0.02 mole) was dissolved in ethanol (230 ml), propylene glycol (190 ml) and water (70.3 ml). Glycolic acid 1.5 g (0.02 mole) was added with stirring to form a molecular complex as shown by decreasing pH of the mixture. The formation of the molecular complex was complete when the pH did not change further. The antifungal formulation thus prepared with pH 4.4 contained 1.6% miconazole and 0.3% glycolic acid in a molecular complex.

### Example 9

Metronidazole 0.75 g (4.4 mmole) was dissolved in 89.25 ml solution prepared from water (40 parts), ethanol (40 parts) and propylene glycol (20 parts), each part by volume. Gluconic acid 50% in water solution, 10 g (25.5 mmole) was added slowly to form a molecular complex between metronidazole and gluconic acid as shown by a change of pH to 2.4. The composition thus obtained contained a molecular complex formed between 0.75% metronidazole and 5% gluconic acid, and was therapeutically effective for topical treatment of acne and rosacea. Alternatively, a gel composition was readily formulated by the addition of a gelling agent such as methyl cellulose or ethyl cellulose at 1 to 2% concentration.

### Example 10

Metronidazole 2.25 g (13.2 mmole) was dissolved in 67.8 ml solution prepared from water (40 parts), ethanol (40 parts), and propylene glycol (20 parts), each part by volume. Gluconic acid 50% in water solution 30 g (76.5 mmole) was added slowly to form a molecular complex between metronidazole and gluconic acid as shown by a change of pH to 2.1. The composition thus obtained contained a molecular complex formed between 2.25% metronidazole and 15% gluconic acid, and was therapeutically effective for topical treatment of acne and rosacea. Alternatively, a cream composition was readily formulated by mixing the above solution with 2 parts of an oil-in-water emulsion. The cream thus obtained contained 0.75% metronidazole in molecular complex with 5% gluconic acid.

### Example 11

Metronidazole 1.71 g (10 mmole) was dissolved in 94.5 ml solution prepared from water (40 parts), ethanol (40 parts), and propylene glycol (20 parts), each part by volume. Glycolic acid 3.8 g (50 mmole) was added slowly to form a molecular complex between metronidazole and glycolic acid as shown by a change of pH to 2.3. The composition thus obtained contained a molecular complex formed between 1.7% metronidazole and 3.8% glycolic acid, and was therapeutically effective for topical treatment of acne and rosacea. Alternatively, a gel composition was readily formulated by the addition of a gelling agent such as methyl cellulose or ethyl cellulose at 1 to 2% concentration.

The invention has been described with reference to particularly preferred embodiments and examples. Those skilled in the art will appreciate that various modifications may be made to the invention without departing from the spirit and scope thereof.

What is claimed is:

1. A composition comprising a molecular complex formed between:
an alkaline pharmaceutical drug and a polyhydroxy acid or its lactone selected from the group consisting of aldonic acids, aldaric acids, alduronic acids, and mixtures thereof; wherein the composition is for topical administration to improve or alleviate cosmetic conditions or dermatological disorders.

2. The composition as claimed in claim 1, wherein the aldonic acid is represented by the following formula.
R(CHOH)ₙCHOHCOOH
where R is H or alkyl group, and n is an integer from 1-6, and where the aldonic acids may exist as stereoisomers as D, L and DL, or R, S and RS forms.

3. The composition as claimed in claim 2, wherein the aldonic acid is selected from the group consisting of 2,3-dihydroxypropanoic acid (glyceric acid), 2,3,4-trihydroxybutanoic acids (stereoisomers; erythronic acid and erythronolactone, threonic acid and threonolactone), 2,3,4,5-tetrahydroxypentanoic acids (stereoisomers; ribonic acid and ribonolactone, arabinoic acid and arabinolactone, xylonic acid and xylonolactone, lyxonic acid and lyxonolactone), 2,3,4,5,6-pentahydroxyhexanoic acids (stereoisomers; allonic acid and allonolactone, altronic acid and altronolactone, gluconic acid and gluconolactone, mannoic acid and mannolactone, gulonic acid and gulonolactone, idonic acid and idonolactone, galactonic acid and galactonolactone, talonic acid and talonolactone), 2,3,4,5,6,7-hexahydroxyheptanoic acids (stereoisomers; alloheptonic acid and alloheptonolactone, altroheptonic acid and altroheptonolactone, glucoheptonic acid and glucoheptonolactone, mannoheptonic acid and mannoheptonolactone, guloheptonic acid and guloheptonolactone, idoheptonic acid and idoheptonolactone, galactoheptonic acid and galactoheptonolactone, taloheptonic acid and taloheptonolactone), and mixtures thereof.

4. The composition as claimed in claim 1, wherein the polyhydroxy acid or its lactone is selected from one or more of the group consisting of gluconic acid, gluconolactone, ribonic acid, ribonolactone, galactonic acid, galactonolactone, glucoheptonic acid, glucoheptonolactone, glucuronic acid, glucuronolactone, galacturonic acid, galacturonolactone, glucaric acid, glucarolactone, galactaric acid, galactarolactone, and mixtures thereof.

5. The composition as claimed in claim 1, wherein the alkaline pharmaceutical drug is selected from the group consisting of acebutolol, acetohydroxamic acid, actiq, acyclovir, albuterol, allopurinol, alloxanthine, alprazolam, alprenolol, amiloride, amantadine, aminacrine, amitriptyline, amorolfine, amodiaquin, amocarzine, amoxapine, atenolol, bemegride, benzocaine, bepridil, benztropine, bupivacaine, bupropion, burimamide, brompheniramine, butoconazole, caffeine, carbamazepine, chlordiazepoxide, chloroquine, chlorpheniramine, chlorpromazine, cimetidine, clonidine, cocaine, codeine, cyclizine, chlorhexidine, citalopram, clemastine, clindamycin, clioquinol, clotrimazole, clozapine, cromolyn, crotamiton, cyclizine, cycloserine, dexmedetomidine, dicyclomine, dihydromorphine, diphenhydramine, diphenoxylate, disopyramide, dobutamine, dopamine, dopamide, dopa esters, doxepin, doxylamine, dyclonine, desipramine, diazepam, dihydrocodeine, diphenoxylate, ephedrine, epinephrine, epinine, ergotamine, econazole, erythromycin, etidocaine, etomidate, fentanyl, fluoxetine, fluphenazine, flurazepam, fluvoxamine, guanethidine, guaifenesin, N-guanylhistamine, haloprogin, hydralazine, hypoxanthine, ichthammol, imiquimod, indomethacin, imipramine, irbesartan, isoetharine, isoproterenol, ketamine, ketanserin, ketoconazole, ketoprofen, kanamycin, labetalol, lamotrigine, lidocaine, lobeline, losartan, loxapine, lysergic diethylamide, mafenide, maprotiline, mecamylamine, meclizine, meclocycline, meperidine, mepivacaine, mescaline, metanephrine, metaproterenol, methadone, methoxamine, metiamide, metolazone, metronidazole, miconazole, midazolam, minocycline, minoxidil, mirtazapine, mupirocin metaraminol, methadone, methamphetamine, methyldopamide, methyldopa esters, metoprolol, mexiletine, molindone, morphine, moxonidine, 3,4-methylenedioxymethamphetamine, nadolol, naftifine, naloxone, nefazodone, neomycin, nifedipine, nystatin, nicotine, norepinephrine, octopamine, olanzapine, ondansetron, oxiconazole, oxotremorine, oxymetazoline, paroxetine, pentazocine, phencyclidine, pheniramine, phenmetrazine, phentolamine, phenylephrine, phenylpropanolamine, phenelzine, phenoxybenzamine, physostigmine, pilocarpine, pimozide, pipamazine, pirenzepine, podophyllin, podofilox, pramipexole, pramoxine, prenalterol, prilocalne, procaine, promethazine propionate, propranolol, protriptyline, pseudoephedrine, pyrethrin, pyrilamine pentazocine, phenylephrine, physostigmine, pilocarpine, pindolol, prazosin, procainamide, procaine, promazine, promethazine, propranolol, pseudoephedrine, pyrimethamine, quetiapine, quinethazone, quinidine, reserpine, risperidone, ritodrine, ropinirole, ropivacaine, salmeterol, scopolamine, selegiline, serotonin, sertindole, sertraline, sotalol, strychnine, sulconazole, sulfadiazine, sulfanilamide, tamsulosin, tazarotene, terbinafine, terconazole, terfenadine, tetracaine, tetracycline, tetrahydrozoline, theobromine, theophylline, thymol, timolol, tioconazole, tizanidine, tocamide, tolnaftate, tranylcypromine, trazodone, triamterene, triazolam, triflupromazine, tripelennamine, triprolidine, terbutaline, thioridazine, tyramine, tolazoline, xanthine, venlafaxine, verapamil and ziprasidone, and mixtures thereof.

6. The composition as claimed in claim 1, wherein the molar ratio of the alkaline pharmaceutical drug to the polyhydroxy acid or its lactone is within the range of from about 1:0.1 to about 1:40.

7. The composition as claimed in claim 1, wherein the molecular weight of the polyhydroxyacid or its lactone form thereof is within the range of from about 50 to about 1000.

8. The composition as claimed in claim 1, further comprising pharmaceutical and other topical agents selected from the group consisting of: those that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; antiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants; hormones; retinoids; gum disease or oral care agents; topical cardiovascular agents; corn, callus and wart removing agents; dipilating agents, and mixtures and combinations thereof.

9. The composition as claimed in claim 1, further comprising one or more additional agents selected from the group consisting of aclovate, acetylsalicylic acid, adapalene, aluminum acetate, aluminum chloride, aluminum hydroxide, aluminum chlorohydroxide, aminobenzoic acid (PABA), aminocaproic acid, aminosalicylic acid, anthralin, ascorbic acid, ascoryl palimate, azelaic acid, bacitracin, beclomethasone dipropionate, benzophenone, benzoyl peroxide, betamethasone dipropionate, betamethasone valerate, calcipotriene, camphor, capsaicin, carbamide peroxide, chitosan, chloroxylenol, ciclopirox, clobetasol propionate, coal tar, dehydroepiandrosterone, desoximetasone, dexamethasone, estradiol, ethinyl estradiol, fluocinonide, fluocinolone acetonide, 5-fluorouracil, griseofulvin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, indomethacin, kojic acid, menthol, methyl nicotinate, methyl salicylate, monobenzone, naproxen, octyl methoxycinnamate, octyl salicylate, oxybenzone, padimate O, permethrin, phenol, piperonyl butoxide, povidone iodine, resorcinol, retinal, 13-cis retinoic acid, retinoic acid, retinol, retinyl acetate, retinyl palmitate, salicylamide, salicylic acid, selenium sulfide, shale tar, sulfur, triamcinolone diacetate, triamcinolone acetonide, triamcinolone hexacetonide, triclosan, undecylenic acid, urea, vitamin E acetate, wood tar, zinc pyrithione, N-acetyl-prolinamide, N-acetyl-lysine, N-acetyl-ornithine, N-acetyl-glucosamine, and mixtures thereof.

10. A method of forming a molecular complex between an alkaline pharmaceutical drug and a polyhydroxyacid or its lactone selected from the group consisting of aldonic acids, aldaric acids, alduronic acids, and mixtures thereof comprising:
dissolving the alkaline pharmaceutical drug and an alkali in a suitable reaction medium to form a free base of the pharmaceutical drug;
optionally separating the free base of the pharmaceutical drug from the reaction medium; and
adding at least one polyhydroxyacid or lactones thereof to the free base in a suitable reaction medium to form a molecular complex.

11. The method as claimed in claim 10, wherein the reaction medium used to form the free base of the pharmaceutical drug is water.

12. The method as claimed in claim 10, wherein the alkali added to the alkaline pharmaceutical drug is an inorganic alkali.

13. The method as claimed in claim 10, wherein the molecular complex is formed when the pH of the reaction medium has changed.

14. The method as claimed in claim 10, wherein the amount of polyhydroxy acid or its lactone form thereof is within the range of from about 0.1 to about 40 moles per mole of pharmaceutical drug.

15. The method as claimed in claim 14, wherein the amount of polyhydroxy acid or its lactone form thereof is within the range of from about 0.5 to about 5 moles per mole of pharmaceutical drug.

16. A method of treating a cosmetic condition or dermatologic indication in a subject comprising topically administering a therapeutically effective amount of the composition as claimed in claim 1 to a subject in need thereof.

17. The method as claimed in claim 16, wherein the pH of the composition is within the range of from about 2.0 to about 7.0

18. The method as claimed in claim 16, wherein the pH of the composition is within the range of from about 3.0 to about 5.0.

19. The method as claimed in claim 16, wherein the composition is in a form selected from the group consisting of lotion, cream, ointment, and gel.

20. The method as claimed in claim 16, wherein the composition additionally includes a cosmetically or dermatologically acceptable excipient.

21. The method as claimed in claim 16, wherein the method treats, heals or prevents a cosmetic condition or dermatological indication.

22. The method as claimed in claim 16, wherein the method treats, heals, or prevents a cosmetic condition or dermatological indication selected from the group consisting cosmetic and clinical signs of changes associated with intrinsic or extrinsic aging; the damages caused by extrinsic factors such as sunlight, air pollution, wind, cold, dampness, heat, chemicals, smoke, cigarette smoking, and radiations including electromagnetic radiations and ionizing radiations; mucosa; skin erythema; inflammation or reaction caused by internal or external factors; and mixtures thereof.

23. The method as claimed in claim 16, wherein the cosmetic condition or dermatological indication is selected from the group consisting of: disturbed keratinization; inflammation; defective syntheses of dermal components; changes associated with intrinsic and extrinsic aging of skin, nail and hair; dryness or looseness of skin, nail and hair; xerosis; ichthyosis; palmar and plantar hyperkeratoses; uneven and rough surface of skin, nail and hair; dandruff; Darier's disease; lichen simplex chronicus; keratoses; acne; pseudofolliculitis barbae; dermatoses; eczema; psoriasis; pruritus; warts; herpes; age spots; lentigines; melasmas; blemished skin; hyperkeratoses; hyperpigmented or hypopigmented skin; abnormal or diminished syntheses of collagen, glycosaminoglycans, proteoglycans and elastin as well as diminished levels of such components in the dermis; stretch marks; skin lines; fine lines; wrinkles; thinning of skin, nail plate and hair; skin thickening due to elastosis of photoaging, loss or reduction of skin, nail and hair resiliency, elasticity and recoilability; lack of skin, nail and hair lubticants and luster; dull and older-looking skin, nail and hair; fragility and splitting of nail and hair, or used as to lighten the skin.

24. The method as claimed in claim 16, wherein the skin changes associated with aging are selected from the group consisting of progressive thinning of skin, fragile skin, deepening of skin lines and fine lines, wrinkles including fine and coarse wrinkles, lusterless skin surface, coarse and uneven skin, loss of skin elasticity and recoilability, blemished and leathery skin, loss of skin lubricating substances, increased numbers of blotches and mottles, nodules, pre-cancerous lesions, pigmented spots and mottled skin, changes in qualities and quantities of collagen and elastic fibers, solar elastosis, decrease in collagen fibers, diminution in the number and diameter of elastic fibers in the papillary dermis, atrophy of the dermis, stretch marks, reduction in subcutaneous adipose tissue and deposition of abnormal elastic materials in the upper dermis, yellowing skin, telangiectatic skin, and older-looking skin.
